(19) United States
(12) Patent Application Publication
(10) Pub. No.: US 2012/0097156 A1
(43) Pub. Date: Apr. 26, 2012

(54) POSITIVE AIRWAY PRESSURE THERAPY MASK HUMIDIFICATION SYSTEMS AND METHODS

(75) Inventors: Bruce R. Bowman, Eden Prairie, MN (US); Steven Bordewick, Shoreview, MN (US); Clint Vilks, Plymouth, MN (US); Jacob Daly, Blaine, MN (US); Eric Allan Becker, Dayton, MN (US)

(73) Assignee: Somnetics Global Pte. Ltd., Singapore (SG)

(21) Appl. No.: 13/145,244

(22) PCT Filed: Feb. 17, 2010
(86) PCT No.: PCT/US10/24454

§ 371 Date: Oct. 24, 2011

Related U.S. Application Data
(60) Provisional application No. 61/153,103, filed on Feb. 17, 2009.

(51) Int. Cl. A61M 16/16; A61M 16/06

(52) U.S. Cl. 128/201.13

(57) ABSTRACT

The present inventions provide positive airway pressure therapy apparatus to humidify the pressurized air delivered to a user during various positive airway pressure therapies and corresponding methods. The positive airway pressure therapy apparatus may be configured to administer one or more positive airway pressure therapies, including: continuous positive airway pressure therapy (CPAP), bi-level positive airway pressure therapy (BPAP), auto positive airway pressure therapy (autoPAP), proportional positive airway pressure therapy (PPAP), and/or other positive airway pressure therapies.

### CROSS-REFERENCE TO RELATED APPLICATIONS

The present application claims benefit and priority to U.S. Prov. Pat. Appl. No. 61/153,103, filed on Feb. 17, 2009 which is hereby incorporated by reference into the present disclosure.

### BACKGROUND OF THE INVENTION

1. Summary of the Invention

The present inventions relate to positive pressurized respiratory therapy and, more particularly, to humidification apparatus and methods for positive pressurize respiratory therapies.

2. Description of the Related Art

During normal spontaneous breathing, the inhaled ambient air is warmed to body temperature and humidified to near saturation by the upper airway before entering the alveoli, reaching an absolute humidity of about 44 mgH₂O/l at a temperature of about 37° C. This is accomplished through the highly vascular nature of the upper airway. During exhalation, the temperature and humidity of the air which was gained during inhalation, is partially given back to the upper airway and the remainder disbursed to the ambient air with the expired air exiting at a temperature of about 32° C. to 34° C. and an absolute humidity of about 27 to 34 mgH₂O/l. In addition to heating and humidifying the inhaled air, the nose and upper airway helps to filter particle and micro organisms before reaching the alveoli.

Positive airway pressure devices, such as CPAP devices, typically deliver pressurized air including air and other breathable gasses to a patient by way of the nose in order to prevent upper airway occlusion during sleep. The pressurized air is typically administered by a mask placed over the user's nose and/or mouth and at a pressure ranging between about 4 cm to 20 cm of water. Positive airway pressure devices have become the devices of choice for the treatment of chronic sleep apnea and snoring. Many variations of positive airway pressure devices are now commercially available.

A typical positive airway pressure device includes a flow generator, a delivery tube and a mask. In various configurations, the mask may fit over the nose and, sometimes the mouth, may include nasal pieces that fit under the nose, may include nostril inserts into the nares, or some combination thereof. The masks frequently include one or more straps configured to secure the mask to the user.

Patients who have obstructive sleep apnea and receive nasal CPAP therapy using ambient air, do not directly bypass the upper airway. In clinical studies, however, many patients report nasal congestion, stuffiness and dryness using CPAP without added humidification. The cause of this symptom has been reported to be due primarily to flow of ambient air through the nose and out of the mouth through mouth leaks. When high one-way flow of air passes through the nose and out the mouth, significant nasal tissue drying occurs and along with it, the release of vasoactive chemicals that act to increase nasal resistance and cause discomfort as a result of dryness of the nose and mouth. While the use of a heated humidifier during CPAP therapy is not considered a requirement, because many patients complain of nasal or oral dryness, added humidification is often used for long-term CPAP use. It is believed that increased compliance can be achieved in some patients because of increased comfort when using added humidification.

It may be beneficial to provide water in the pressurized air delivered to the user for therapeutic reasons and also for the comfort of the user. Accordingly, positive airway pressure apparatus may include a humidifier. The humidifier is frequently integrated into the flow generator. Some humidifiers are configured such that the flow generator blows pressurized air over a water reservoir in the flow generator. The pressurized humidified air is then conveyed to the mask through the delivery tube. Typically, the water reservoir must have a large surface area so that a large water reservoir must be provided in the flow generator. In addition, the humidified pressurized air may cool as it passes from the flow generator to the user, which may result in condensation in the delivery tube. A buildup of condensation in the delivery tube may increase flow resistance. In some instances, the buildup of condensation may occlude delivery of pressurized air and in other instances the condensation buildup may be directed through the tubing and mask into the airways of a patient during sleep. This can be both uncomfortable and dangerous. Therefore, a need exists for a positive airway pressure device that may avoid or reduce condensation of water within the delivery tube.

Many prior humidification systems use a heating element to heat the water in the reservoir in order to assist in the humidification of the pressurized air and to warm the inhaled air to increase user comfort. However, heating elements consume high levels of power. Modern positive airway pressure devices are being designed to use less energy and, in some cases, to run on battery power as a backup or primary power source. Therefore, a need exists for a positive airway pressure device that may reduce the power required to humidify the air delivered to a patient receiving a positive airway pressure therapy.

In addition, prior humidification systems typically required reservoirs, heating elements and various other integrated components adding to the cost and frequently adding significant size and weight to the CPAP system. The increased size, weight and, in some cases, number of components can be a significant disadvantage, especially when travel portability is important to the user. Therefore a need exists to provide heat and moisture to the air delivered to a patient receiving positive pressure therapy without adding significant cost, size, and weight to the therapy device.

In addition, the reservoirs and other moistened components can foster the growth of bacteria and fungi that can be detrimental to the user. The warm moist environment as well as the complex components of the CPAP system may tend to create environments to foster the growth of bacterial and fungi, as well as other microorganisms. Therefore a need exists for an apparatus and method for moistening and heating air that does not foster the development of bacteria, fungi or other microorganisms.

In addition, the atmospheric air that is pressurized in positive airway pressure therapies may include various irritants, allergans, and/or pathogens that are directed into the airways of a user. The blowers of CPAP systems may tend to draw in these various airborne particulates that may be detrimental to a user. Therefore a need exists for an apparatus and method for moistening and heating air that may inhibit the introduction of these irritants, allergans, and/or pathogens into the airways of a user.

### SUMMARY OF THE INVENTION

Apparatus and methods in accordance with the present inventions may resolve many of the needs and shortcomings discussed above and will provide additional improvements and advantages that may be recognized by those skilled in the art upon review of the present disclosure.

In one aspect, Apparatus in accordance with the present inventions can recover a significant portion of the heat and moisture lost through exhaled air through use of a Heat and Moisture Exchanger (HME). The apparatus may use the HME to, among other functions, act an “artificial nose” supplementing the nose of a user and allowing for similar processes of heat and moisture retention and exchange as well as filtering in certain aspects. An HME used in accordance with the present inventions may be configured with a wide range of performance characteristics depending on the needs of the user by varying the device's heat and moisture properties and its size and configuration.

In certain aspects, HME's used in accordance with the present inventions may offer many advantages over other means of humidifying. These may include being passive devices that can require no external power, can have no moving parts to wear out, and can be less expensive than heated humidifiers.

HME's used in accordance with the present inventions may have an additional potential benefit over traditional heated humidifiers. Heated humidifiers typically produce condensation in the ventilator circuit due to differences in temperatures between ambient air and the circuit air temperature. With the HME functioning as a passive device generally at the distal end of a breathing circuit, the heated and humidified air from the HME travels such a short distance before entering the nose or mouth, that it does not have time to cool and condense to any significant degree. This can reduce the condensation in the circuit which can very easily become contaminated.

Apparatus in accordance with various aspects of the present inventions may be configured as a positive airway pressure therapy apparatus. The positive airway pressure therapy apparatus may include a flow generator that has an outlet. The flow generator is generally configured to provide pressurized air at the outlet. The positive airway pressure therapy apparatus may further include a user interface. The user interface includes a mask and support bands. The mask includes a heat and moisture exchange element between a vent and the mask outlet. The mask may further include one or more sensors in fluid communication with the chambers or tubing of the mask between the HME element and the mask outlet.

The present inventions include methods for administering positive airway pressure therapies including using passive humidification in the mask. The methods may also include sensing the pressure and/or flow in the chambers or tubing of a mask downstream of a Heat and Moisture Exchange (HME) element and regulating and/or controlling the blower pressure based on the sensed pressure or flow. The methods may include the regulation of a positive airway pressure apparatus

Other features and advantages of the invention will become apparent from the following detailed description and from the claims.

### BRIEF DESCRIPTION OF THE DRAWINGS

FIG. 1A illustrates a perspective view of an exemplary embodiment of a positive airway pressure apparatus in accordance with aspects of the present inventions;

FIG. 1B illustrates a cross-sectional view through section line 1B-1B of the embodiment of FIG. 1A;

FIG. 1C illustrates a detailed perspective view of the embodiment of FIG. 1A;

FIG. 2 illustrates a front view of another exemplary embodiment of a positive airway pressure apparatus having nares seals in accordance with aspects of the present inventions;

FIG. 3 illustrates front view of another exemplary embodiment of a positive airway pressure apparatus having a face mask in accordance with aspects of the present inventions;

FIG. 4A illustrates front view of another exemplary embodiment of a positive airway pressure apparatus having a nose mask in accordance with aspects of the present inventions

FIG. 4B illustrates a perspective view of the embodiment of FIG. 4A of a portion of the user interface with the mask removed;

FIG. 4C illustrates a cross-sectional view of the embodiment of FIG. 4A through the frontal plane;

FIG. 5A illustrates a front view in partial cross-section of another exemplary embodiment of a positive airway pressure apparatus in accordance with aspects of the present inventions;

FIG. 5B illustrates a cross-sectional view through section line 5B-5B of the embodiment of FIG. 5A;

FIG. 6A illustrates an elevated perspective view of an exemplary embodiment of a portion of the user interface defining the HME cavity within the air passage divided into an interlocking upper section and lower section;

FIG. 6B illustrates an downward perspective view of a portion of the user interface defining the HME cavity within the air passage divided into an interlocking upper section and lower section of an embodiment of the present invention similar to that of FIG. 6A;

FIG. 7A illustrates an elevated perspective view of an exemplary embodiment of an HME element secured within an HME cavity;

FIG. 7B illustrates an elevated perspective view of another exemplary embodiment of an HME element secured within an HME cavity;

FIG. 7C illustrates an elevated perspective view of another exemplary embodiment of an HME element secured within an HME cavity;

FIG. 7D illustrates an elevated perspective view of the exemplary embodiment of FIG. 7C of an HME element secured within an HME cavity;

FIG. 8A illustrates a front view of an exemplary embodiment of a portion of the user interface defining the HME cavity within the air passage having an upper section interlocked with a lower section;

FIG. 8B illustrates a side view of an exemplary embodiment of a portion of the user interface defining the HME cavity within the air passage having an upper section interlocked with a lower section;

FIG. 8C illustrates a cross-sectional view through section lines E-E of the embodiment of FIG. 8B a portion of the user interface defining the HME cavity within the air passage having an upper section interlocked with a lower section;

FIG. 8D illustrates an elevated perspective view of the upper section of the exemplary embodiment of FIGS. 8A to 8C of an HME element secured within an HME cavity;

FIG. 9A illustrates a front view of an exemplary embodiment of a portion of the user interface defining the HME cavity within the air passage having an upper section interlocked with a lower section;

FIG. 9B illustrates a side view of an exemplary embodiment of a portion of the user interface defining the HME cavity within the air passage having an upper section interlocked with a lower section;

FIG. 9C illustrates a cross-sectional view through section lines F-F of the embodiment of FIG. 9B of a portion of the user interface defining the HME cavity within the air passage having an upper section interlocked with a lower section;

FIG. 9D illustrates an elevated perspective view of the upper section of the exemplary embodiment of FIGS. 9A to 9C of an HME element secured within an HME cavity;

FIG. 10A illustrates a front view of an exemplary embodiment of a portion of the user interface defining the HME cavity within the air passage having an upper section interlocked with a lower section;

FIG. 10B illustrates a side view of an exemplary embodiment of a portion of the user interface defining the HME cavity within the air passage having an upper section interlocked with a lower section;

FIG. 10C illustrates a cross-sectional view through section lines G-G of the embodiment of FIG. 10B of a portion of the user interface defining the HME cavity within the air passage having an upper section interlocked with a lower section;

FIG. 10D illustrates an elevated perspective view of the upper section of the exemplary embodiment of FIGS. 10A to 10C of an HME element secured within an HME cavity;

FIG. 11 illustrates a side view in partial cross-section of another exemplary embodiment of a positive airway pressure apparatus in accordance with aspects of the present inventions;

FIG. 12 illustrates an exemplary control diagram for regulation of an apparatus in accordance with aspects of the present invention;

FIG. 13A illustrates an elevated perspective view of an exemplary embodiment of an HME element in accordance with aspects of the present invention;

FIG. 13B illustrates top view of an exemplary embodiment of an HME element in accordance with aspects of the present invention;

FIG. 13C illustrates cutaway side view along line 13C-13C of FIG. 13B of the HME element in accordance with aspects of the present invention;

FIG. 14A illustrates an elevated perspective view of an exemplary embodiment of an HME element in accordance with aspects of the present invention;

FIG. 14B illustrates top view of an exemplary embodiment of an HME element in accordance with aspects of the present invention; and

FIG. 14C illustrates cutaway side view along line 14C-14C of FIG. 14B of the HME element in accordance with aspects of the present invention.

All Figures are illustrated for ease of explanation of the basic teachings of the present invention only; the extensions of the Figures with respect to number, position, relationship and dimensions of the parts to form the embodiment will be explained or will be within the skill of the art after the following description has been read and understood. Further, the exact dimensions and dimensional proportions to conform to specific force, weight, strength, flow and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they generally correspond with airflow within an apparatus in accordance with the present inventions.

### DETAILED DESCRIPTION OF THE INVENTION

The present inventions provide positive airway pressure therapy apparatus 10 and associated methods for treatment of sleep apnea and other respiratory and sleeping disorders. The positive airway pressure therapy apparatus 10 are typically configured to communicate pressurized air to a user lying in bed from a remotely positioned flow generator 20. The positive airway pressure therapy apparatus 10 may include a flow generator 20, a Heat and Moisture Exchange (HME) element 70, and a user interface 40. In certain aspects, the positive airway pressure therapy apparatus 10 may also include a delivery tube 30. The flow generator 20 is generally configured to provide pressurized air at pressures adequate to administer positive airway pressure therapy to a user. When present, the delivery tube 30 is configured to communicate pressurized air from the flow generator 20 to the user interface 40. The user interface 40 is configured to communicate the pressurized air from the flow generator 20 into the airways of a user. Typically, the user interface 40 is configured to be secured relative to the user's head such that a positive pressure therapy may be administered to a user during therapy. The HME element 70 is generally configured to capture the heat and moisture from the air exhaled from the user and return at least some of the recovered heat and moisture into the air from the flow generator to be inhaled by the user. The HME element 70 may, in some aspects, be generally disposed between vents 80 and the mask outlet 69. One or more sensors 100 may be configured to sense the pressure or airflow in the mask chamber 66 between the HME element 70 and the mask outlet 69 when the mask 60 is sealingly engaged against a user.

The Figures generally illustrate exemplary embodiments of positive airway pressure therapy apparatus 10 in accordance with aspects of the present inventions. The particularly illustrated embodiments of the positive airway pressure therapy apparatus 10 have been chosen for ease of explanation and understanding of various aspects of the present inventions. These illustrated embodiments are not meant to limit the scope of coverage but, instead, to assist in understanding the context of the language used in this specification and in the appended claims. Accordingly, the appended claims may encompass variations of the present inventions that differ from the illustrated embodiments.

Positive airway pressure therapy apparatus 10 in accordance with aspects of the present invention include at least a mask 60 and an HME element 70. A flow generator 20 may also be provided and configured to provide one or more positive airway pressure therapies to a user through the HME element 70 and mask 60. The one or more positive airway pressure therapies may include continuous positive airway pressure therapy (CPAP), auto positive airway pressure therapy (autoPAP), and/or other positive airway pressure therapies as will be recognized by those skilled in the art upon review of this disclosure.

The flow generator 20 typically includes a flow generator housing 22 having an outlet 24, with the flow generator 20 adapted to deliver pressurized air to the outlet 24. In order to deliver pressurized air to the outlet 24, the flow generator 20 may include one or more of various motors, fans, pumps, turbines, ducts, inlets, conduits, passages, mufflers, and other components, as will be recognized by those skilled in the art upon review of the present disclosure. A control unit 26 may be included in the positive airway pressure therapy apparatus 10.

The control unit 26 may be adapted to control one or more components of the flow generator 20. The control unit 26 may be configured to detect phases of the user's breathing cycle and/or to regulate the flow generator 20 and other components to administer one or more of a variety of positive airway pressure therapies that will be recognized by those skilled in the art upon review of the present disclosure. The control unit 26 will typically be positioned within or on the flow generator housing 22, but may be otherwise positioned or located, including remotely, as will be recognized by those skilled in the art upon review of the present disclosure. The control unit 26 is operably connected to one or more components of the flow generator 20. The control unit 26 may include one or more circuits and/or may include one or more microprocessors as well as a computer readable memory.

The control unit 26 is typically configured to output one or more control signals to various components of the flow generator 20 and other components of the positive airway pressure therapy apparatus 10. The control unit 26, in some aspects may be adapted to receive one or more signals from one or more components of the positive airway pressure therapy apparatus 10. The control unit 26 may process or otherwise utilize the signals from the components of the positive airway pressure therapy apparatus 10 in formulating the one or more control signals output to various components. The control unit 26 may be particularly adapted to control the flow generator 20 in response to signals from sensor 100. The control unit 26 may be further adapted to control other components of the positive airway pressure therapy apparatus 10.

In one aspect, the control unit 26 may control the flow generator 20 in response to information including commands from the control interface 28. The control interface 28 may include one or more buttons, switches, touch screens, or other controls for controlling the flow generator 20 and associated components. The control interface 28 may be in communication with the control unit 26 to transfer information to and from the control unit 26. Portions of the control interface 28 may be mounted on the flow generator housing 22 or may be otherwise positioned on components of the apparatus 10 or remotely as will be recognized by those skilled in the art upon review of the present disclosure.

The user interface 40 is generally configured to be secured to a user and to communicate pressurized air into the airway of a user. Typically, the user interface 40 will include at least a mask 60, and one or more support bands 44 to secure the mask 60 to a user. The user interface 40 may define at least a portion of an air passage 74 that extends between the flow generator 20 and the mask outlet 69 to communicate pressurized air from the flow generator to the airways of the user. The user interface 40 may also include various features such as pads that allow the user interface 40 to be securely and comfortably affixed to the user and that maintain a proper orientation of the user interface 40 with respect to the user.

The mask 60 may be configured to sealingly engage the user to permit the communication of pressurized air generated to the user's airways. In various aspects, portions of the mask 60 may be positioned about the user's nose, the user's mouth, or both the user's nose and mouth and within the nares of the user in order to provide a generally sealed connection to the user's airway for the delivery of pressurized air for at least the inhalation portion of a breath. In some aspects, the mask 60 may include nasal pieces that fit in, around and/or under the user's nose, including in some embodiments nostril inserts that are received in the nares with seals 76 that sealingly engage the user's nares or nose. A pressure greater than atmospheric pressure may be continuously or variably provided within the sealed connection. Accordingly, portions of the mask 60 may be formed of soft silicone rubber or similar material that may provide a seal 76 and that may also be generally comfortable when positioned against the user's skin.

The mask 60 may include an interior mask surface 64. At least a portion of the interior mask surface 64 defines a mask chamber 66. In other aspects, the interior mask surface 64 may define at least a portion of a chamber 66 when generally sealed about portions of the user's face. The mask 60 typically includes one or more mask inlets 68 through which pressurized air may be communicated into the chamber 66 and through one or more mask outlets 69 to communicate the pressurized air to the airway of the user. In some aspects, pressurized air may be inhaled from the chamber 66 by the user.

In some aspects, the user interface 40 may include the flow generator 20 such that the flow generator 20 is generally secured about the user's head. The flow generator 20 may communicate within an air passage 74 defined by the user interface 40 or defined by a delivery tube 30 of the user interface 40 to convey pressurized air to the mask 60 for the user's inhalation. In other aspects, the flow generator 20 may be a separate component and connected to the user interface 40 by a delivery tube 30. The delivery tube 30 when present forms at least a portion of the air passage 74. The delivery tube 30 may be secured to an outlet 24 of the flow generator 20 to convey pressurized air from the flow generator 20 to the user interface 40. In one aspect, the delivery tube 30 may be configured as an elongated flexible tube. The delivery tube 30 may be composed of a lightweight plastic, and often has a ribbed configuration. The proximal end of the delivery tube 30 may be adapted to be secured to the flow generator 20 with the air passage 74 in fluid communication with the outlet 24 of the flow generator 20. The user interface 40 may be secured to the distal end of the delivery tube 30 in fluid communication with the air passage 74. Accordingly, pressurized air from the flow generator 20 may be conveyed into the air passage 74 of the delivery tube 30 and delivered to the user interface 40. The distal end of the delivery tube 30 is typically connected to the user interface 40 at a connector.

One or more vents 80 may be provided to release pressurized air from the air passage 74 into the ambient atmosphere. In certain aspects, the vents 80 may allow a small continual flow of air from the flow generator 20 down the delivery tube 30 through air passage 74 and out vents 80 into the ambient atmosphere because of the pressure differential between the external atmosphere and the air passage 74. This continual flow through air passage 74 and vents 80 can insure that at a significant portion of the CO₂ that may not be initially purged through vents 80 during exhalation and is, instead, directed proximally (toward the flow generator 20) into air passage 74, is purged from the air passage 74 through vents 80 before and/or during the next inhalation such that re-breathing of significant amounts of exhaled CO₂ may be avoided. At therapeutic pressures, the vents 80 may be configured to the release of at between about 10 liters per minute to about 60 liters per minute. For example, a therapy administering a pressure of 4 centimeters of water could require a vent 80 permitting a rate of airflow of between 12 to 30 liters per minute from the air passage 74. In another example, a therapy administering a pressure of around 20 centimeters of water could require a vent 80 permitting a rate of airflow of between 30 to 50 liters per minute. The vents 80 may be in the form of holes, louvers, slots, valves and other passages or structures as will be recognized by those skilled in the art upon review of the present disclosure. The vents 80 may be configurable in one or more of an open or closed position by various electrical or mechanical actuators that may be controlled by the control unit 26. In one aspect, the one or more vents 80 may be included in the mask 60. In other aspects, the one or more vents 80 may be included in the user interface 40 or delivery tube 30. The vents 80 are typically configured to vent off the majority of CO₂ that may have been directed proximally up the delivery tube 30 without wasting power and flow generator 20 capacity by blowing air into the atmosphere unnecessarily. Typically, the vents 80 are positioned on the proximal side of the HME element 70 at a location near or adjacent to the HME element to minimize the dead space between the HME element 70 and the vents 80. In configuring vents 80, consideration should be made as to the minimum pressure therapy that will be administered and factors such as expected tidal volume, maximum flow rate and breath rate that a user may experience during therapy as well as the volume of airspace between the nose or mouth and the vents 80 as will be recognized by those skilled in the art upon review of the present disclosure. These may be determined through use of the test methods for measuring CO₂ re-breathing as described in ISO International Standard 17510-2 section 5.3.

The HME element 70 is generally configured to collect moisture and heat from expired air and to reintroduce at least a portion of the moisture and heat into the air inhaled from the flow generator 20 during the next breath. The HME element 70 is generally positioned at least between vents 80 and the mask outlet 69. This relative positioning may permit the air that is not inhaled from the flow generator 20 to be directed out the vents 80 without passing through the HME element 70. The venting of this air prior to passage through the HME element 70 will prevent this air from drying and cooling of the heat and moisture retained by the HME element 70 from a prior exhalation. Thereby, the retained heat and moisture in the HME element 70 from the previous exhalation can be made available for transfer into the air of next inhaled breath. During exhalation, the warm high humidity air passes over the cooler dryer material of the HME element 70 and causes moisture to condense on the material and to warm the material. The reverse happens on inspiration where ambient air is cooler and dryer than the material of the HME element 70 and the heat and moisture from the material is exchanged to the inspired air. In certain aspects, the HME element 70 may be further configured to capture particulate materials. During inhalation, this may prevent the introduction of various particulate materials into the airways of the user. During exhalation, this may prevent the spreading of various pathogens among other things.

The HME element 70 is generally configured to permit the flow of air and to collect heat and water from expired air to warm and humidify the gas subsequently inspired by the user. In certain aspects, the HME element 70 may be configured to transfer water with an efficiency of at least 5 mg H₂0 per liter of air. In some aspects, the HME element 70 may be configured to transfer water with an efficiency of between 10 and 33 mg H₂0 per liter of air. The HME element 70 is typically made from one or more of three types of materials. These materials may include 1) a hydrophobic material which typically has high antimicrobial properties but may not be as effective in retaining and transferring humidity, 2) a hygroscopic material which typically has much better humidifying effectiveness but may lack microbial filtration properties, and 3) a combination of a hydrophobic material and a hygroscopic material, which may provide both good humidity retention and transfer as well as antimicrobial properties. The material(s) may be formed into various sheets, fibers, foams or other forms which may be arranged in one or more layers or other configurations. The material may be one or more of a cellulose based material, a polysulphone, an electrostatically charged polypropylene, polycarbonate or other materials as will be recognized by those skilled in the art upon review of the present disclosure. In certain aspects, the HME element 70 may include an electrostatic filter to assist in the trapping of bacteria and/or viruses. The material may be configured as a set of random or organized intermeshed fibers, a foam or a sheet or in some aspects an array of small parallel tubes. The material can be configured to function as a filter or may include additional filtering components that may be configured to remove particulates including various virus and bacteria.

In certain aspects, the HME may be configured from paper or foam impregnated with a moisture-absorbing hygroscopic salt such as AlCl₃, CaCl₂, MgCl₂, or LiCl which enhances their ability to retain moisture. This configuration of HME element may be referred to as a hygroscopic condenser humidifier. The material may also be treated with various hydrophilic materials such as polyacrylic acid, polyvinyl pyrrolidone, polyvinyl alcohol, or other hydrophilic polymers, glycol or glycerine to enhance its efficiency as will be recognized by those skilled in the art upon review of the present disclosure.

The material is typically incorporated into the HME element 70 in a manner which may maximize surface area and reduce resistance to airflow. When formed as a sheet, the material which forms the HME element 70 is typically folded into pleats or rolled to maximize surface area and reduce resistance to the passage of air. However, HME elements 70 may take a wide variety of forms and be formed from a wide variety of materials and combinations of materials as will be recognized by those skilled in the art upon review of the present disclosure.

In certain aspects, the HME element 70 can be formed from one or more layers of aluminum configured for a maximum surface area that comes into contact with the airflow created by respiration in a way that also minimizes resistance to the airflow. On exhalation, moisture condenses on the cooler aluminum sheets while warming the aluminum. On inhalation, the warm, moist aluminum sheets transfer heat and moisture to the incoming air.

In other aspects, the HME element 70 can be moisture absorbing heat-insulating disk-like sheets that are alternately interspersed with permeable heat-conductive disk-like sheets. The heat-insulating sheets may be made of gauze or non-woven fabric. The heat-conductive sheets may be made of a mesh of metal such as aluminum. The heat conductive sheets may be oriented perpendicular to the direction of airflow. In another similar aspect, the heat and moisture exchange material may be spirally wound using aluminum mesh with gauze to allow for the free flow of air across the aluminum winding.

In still other aspects, the HME element 70 may be formed from a folded sheet material and strip material where the sheet material is folded with parallel sharp folds with each new fold slightly staggered. The sheet material may be configured to be permeable to air but impermeable to bacteria. Such sheet materials may be either hydrophobic or hydrophilic and may include a matrix of hydrophobized compressed glass fibers. The strip material may be further configured as strips of micro-corrugated paper which is permeable to air and other hydrophilic materials which have good heat and moisture exchange capacity. These materials can, for example, be in the form of foamed polyurethane, loosely packed polyethylene, or polyester fibers. Other materials that may be used can include cardboard or paper, glass fibers, or cellulose within a waved or plane surface. These and other configurations of the material of HME element 70 will be recognized by those skilled in the art upon review of the present disclosure.

The HME element 70 is generally secured within the air passage 74 such that both exhaled air and inhaled air are directed through the HME element 70 during a breath cycle of a user. A particular HME element 70 may be selected based on its efficiency at retaining one or both of heat and moisture. Therefore, use of different HME elements 70 with either higher or lower efficiency in retaining and exchanging heat and moisture may be desirable.

The HME element 70 may be positioned within an HME cavity 75 defined along at least a portion of the length of the air passage 74. The HME element 70 may be positioned distal to the vent 80 so that the constant flow of air from the flow generator 20 down the delivery tube 30 through the air passage 74 and out vents 80 does not flow through HME 70.

The HME cavity 75 is generally configured to retain the HME element 70 and to prevent air leaks around the HME element 70. The HME cavity 75 is generally formed between the vents 80 and the mask outlet 69. The HME cavity 75 may be configurable in both of an open position and a closed position to permit the replacement of an HME element 70 secured in the HME cavity 75. The HME cavity 75 may be an area of enlarged cross-sectional area to permit a larger diameter HME element 70 to be positioned in the HME cavity 75. The HME cavity 75 may also be a portion of the air passage 74 having the same or smaller diameter than the adjacent air passage diameter. Various adapters (not shown) may also be provided to accommodate different sizes and types of HME elements 70 in a particular HME cavity 75. An adapter may permit different configurations of HME elements 70 to be fitted within the HME cavity 75. This may permit a user to select the HME element 70 having the desired properties of airflow, heat and moisture exchange capacities, as well as other characteristics that will be recognized by those skilled in the art upon review of the present disclosure. The HME element 70 may be secured at a desired location within the HME cavity 75 with an element lock 77. The element lock 77 may take the form of various clips, detents, fasteners, perforated plates, seals, and the like that secure the HME element 70 at the desired location during operation. The element lock 77 is typically configured to minimally restrict airflow across the HME element 70 and through the air passage 74. In other aspects, the HME element 70 may be secured to at least a portion of the interior mask surface 64 and extend along a length of the air passage 74 within the mask 60.

One or more sensors 100 may be provided to sense the pressure and/or the airflow between at least the HME element 70 and the mask outlet 69 during therapy. Additional sensors may be located proximal to the HME element 70 as will be recognized by those skilled in the art. The one or more sensors 100 may be configured to communicate at least data indicative of pressure and/or airflow to the control unit 26. The one or more sensors 100 may be configured to measure the pressure and/or the airflow from a location distal to the HME element 70. The positioning of the one or more sensors 100 distal to the HME element 70 may permit a more accurate measure of therapeutic pressure being administered to the patient. The one or more sensors 100 may permit the therapy to be adapted during administration of a therapeutic session as resistance to airflow through the HME element 70 varies over time. The airflow through the HME element 70 may vary due to a number of factors including changes in temperature, moisture content, amount of condensed water covering the HME material, accumulation of debris, aging or otherwise as will be recognized by those skilled in the art upon review of the present disclosure.

The one or more sensors 100 may generally be positioned or other wise configured to receive a pressure signal and/or airflow signal from a position between the HME element 70 and the mask outlet 69. The sensor 100 may be positioned within the air passage 74. The sensor 100 may be positioned in close proximity to the nose or mouth to increase the responsiveness of the sensor 100 in generating a signal indicative of pressure and/or airflow. In this aspect, the one or more sensors 100 may transmit data to the control unit 26 through a wired or wireless connection 101 between the one or more sensors 100 and the control unit 26. In another aspect, at least one of the one or more sensors 100 may be in fluid communication with a sensor tube 98. The sensor tube 98 defines a sensor tube passage 99. The sensor tube 98 is configured to be in fluid communication with the air passage 74 at a location between the HME element 70 and the mask outlet 69 at a distal end of the sensor tube 98. The sensor tube passage 99 is also in fluid communication with the sensor 100. The sensor 100 may be positioned at a proximal end of the sensor tube 98 or otherwise in communication with the sensor tube passage 99 at a location at or near the distal end of the sensor tube 98. In this aspect, the sensor 100 may be positioned remotely from the user interface 40. In certain aspects, the sensor 100 may be positioned adjacent the control unit 26 or may be integral with the control unit 26. In other aspects, the sensor 100 may be positioned in the flow generator housing 22.

Support bands 44 are configured to secure the user interface 40 and/or mask 60 to the user. The support bands 44 are typically in the form of elongated members that are configured to exert sufficient tension to retain the interface conduit 50 on the head of the user and, more particularly, to retain the mask 60 generally oriented to deliver pressurized air to the user as the user sleeps. In certain aspects, the support bands 44 are configured as flattened straps to comfortably distribute a force over their surface area. The support bands 44 may be formed from one or more stretchable elastic materials, substantially unstretchable materials, or other materials as will be recognized by those skilled in the art upon review of the present disclosure. The support bands 44 may be integrally formed or interconnected by a variety of mechanical linkages. The support bands 44 may incorporate various buckles, snaps, hook and loop type fasteners, or other components to link and/or permit relative adjustment of the support bands 44. Various aspects of the support bands 44 may be adjustable by the user to position, inter alia, the interface conduit 50 and mask 60. These aspects may include length, relative positions or other aspects as will be recognized by those skilled in the art upon review of the present disclosure.

In operation, the user interface 40 may be secured to the user's head and the mask 60 positioned in communication with the user's nose and/or mouth. The flow generator 20 generates a flow of pressurized air that is directed through the air passage 74. Upon inhalation of the user, air flows from the flow generator 20, down the delivery tube 30, and out vents 80 as well as past vents 80 and through the HME element 70 to the user. The one or more sensors 100 may measure the pressure and/or the airflow of the pressurized air distal to the HME element 70 and the data may be transmitted to the control unit 26. The pressurized air continues through the air passage 74 defined by the mask 60, out the mask outlet 69 and into the airway of a user. Upon exhalation, the airflow reverses through a portion of the air passage 74. The air from the user moves from the airway of the user through the mask outlet. The pressure and/or airflow at exhalation may be measured by the one or more sensors 100 and the data may be transmitted to the control unit 26. The air passes through the HME element 70 where at least some of the moisture and heat from the exhaled air are retained. After passing the HME element 70, most of the exhaled air is typically vented through vents 80, but some may be blown proximally up air passage 74. By the time of the next inhalation, at least a portion of this proximally blown air has been directed distally through the air passage 74 and out the vents 80, thus purging the air passage 74 of exhaled CO₂ from the user.

Based upon at least some of the data provided by the one or more sensors 100, the control unit 26 may adjust the pressure produced by the flow generator 20 or other parameters such as for example variations in pressure from the flow generator 20 over the breath cycle of the therapy being administered to the user. Upon a subsequent breath, the pressurized air from the flow generator 20, the air flows out vents 80 to ambient as well as past vents 80 and through the HME element 70 to the user. The HME element 70 may introduce at least a portion of the moisture and heat captured from prior inhalations. The one or more sensors 100 may again measure the pressure and/or the airflow of the pressurized air distal to the HME element 70 and the data may be transmitted to the control unit 26. The control unit 26 may adjust the therapy based on variations in resistance to the passage of air through the HME element 70 or otherwise as dictated by the user's therapy.

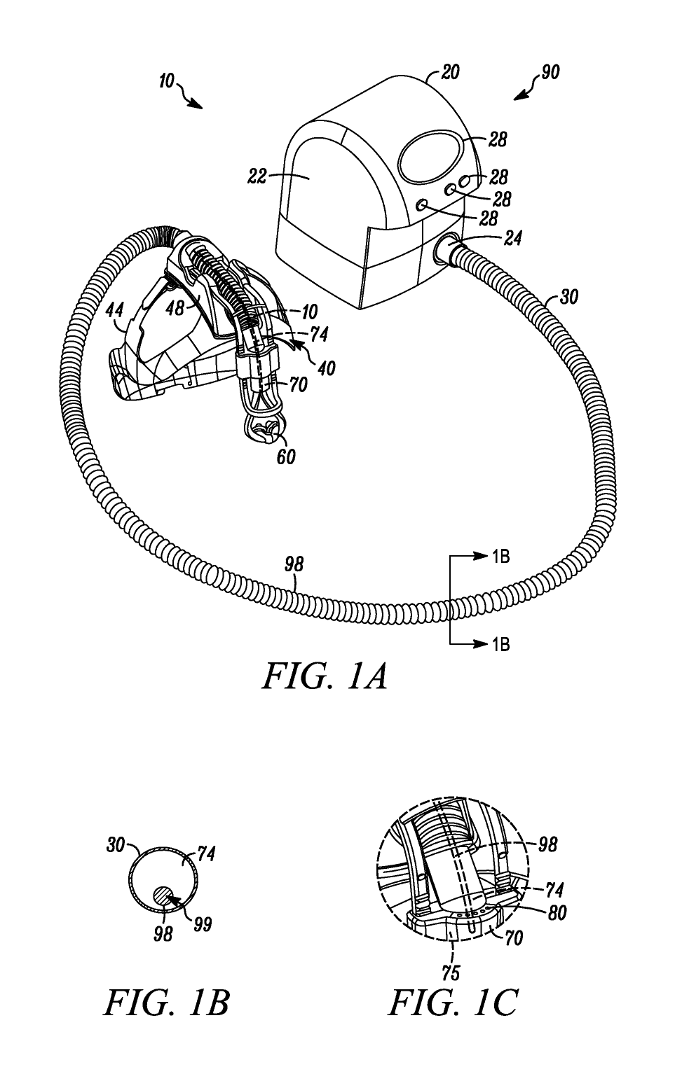

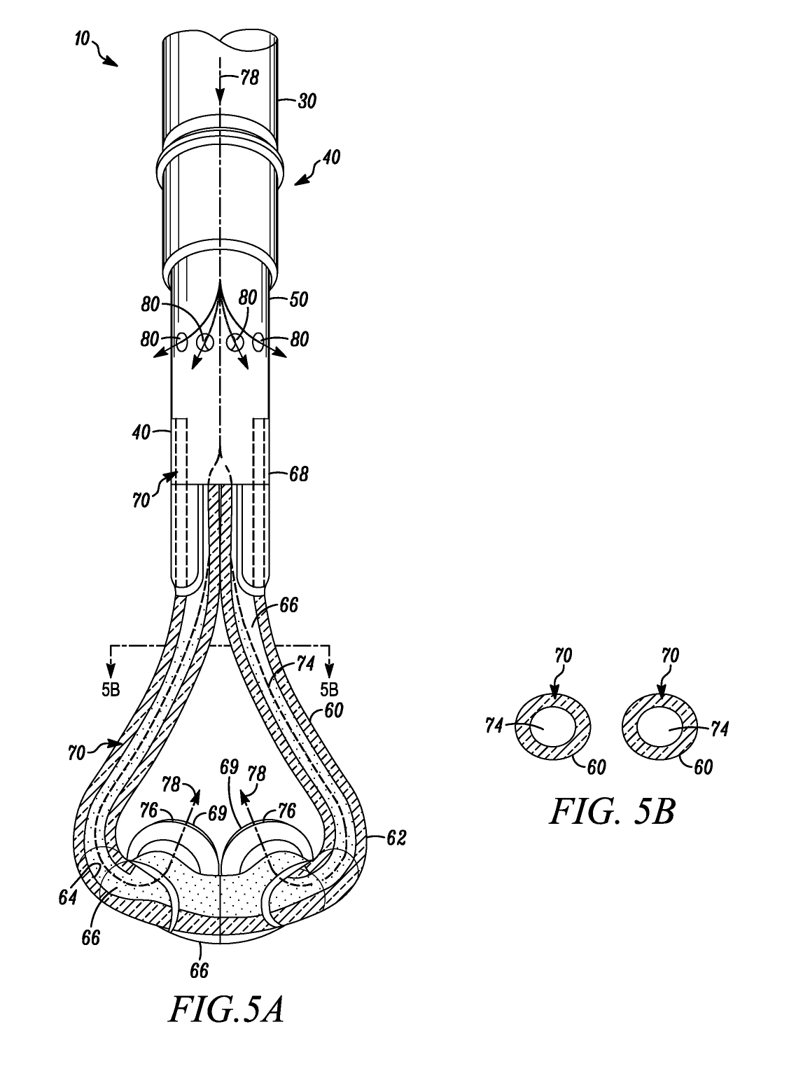

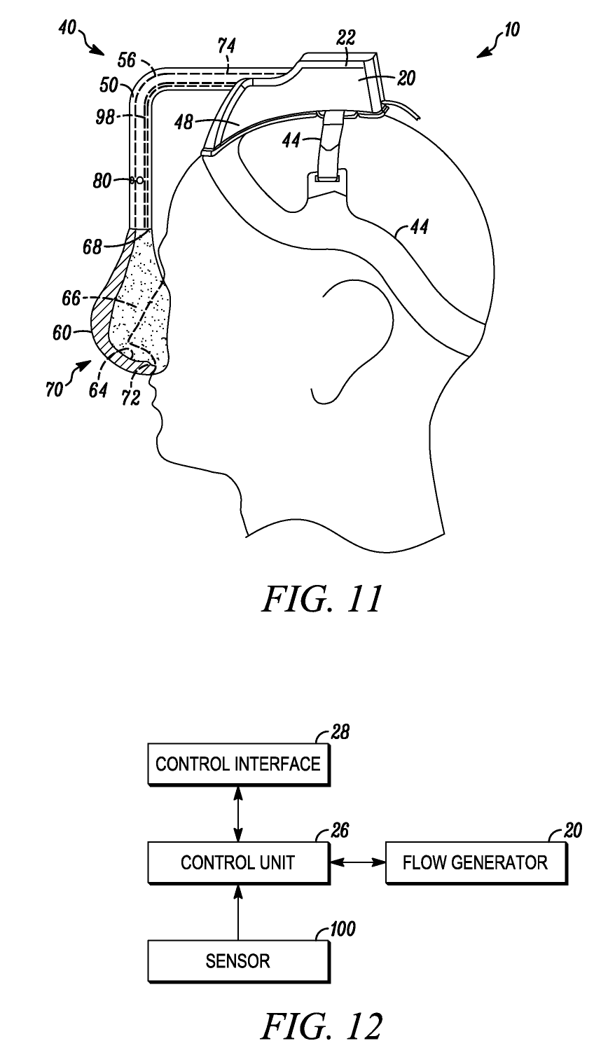

FIGS. 1A to 1C generally illustrate an exemplary embodiment of a positive airway pressure therapy apparatus 10 in accordance with aspects of the present inventions. As illustrated, the positive airway pressure therapy apparatus 10 includes a flow generator 20, a delivery tube 30, and a user interface 40. The user interface 40 defines the air passage 74, vents 80 and HME cavity 75. The flow generator 20, in this embodiment, is remote from the user interface 40. The proximal end of the delivery tube 30 is attached to a pressurized air outlet 24 of the flow generator 20. A sensor tube 98 extends through the air passage 74 defined by the delivery tube 30. In one aspect, this reduces the number of external tubes and the likelihood of entanglement or snagging as a user sleeps. An exemplary cross-section of the delivery tube 30 is illustrated in FIG. 1B, which shows the relationship of the sensor tube 98 extending through the air passage 74. The air tube 98, in this embodiment, may be adapted to communicate the pressure from a distal end of the air tube 98 to a sensor 100 positioned at a proximal end of the air tube 98 within the flow generator housing 22. In other embodiments, the air tube 98 may be external to the delivery tube 30, and may be secured to the delivery tube 30 by, for example, various snaps, clips, and pre-molded hooks or clips. FIG. 1C illustrates, in phantom, the air delivery tube 98 extending through a portion of the air passage 74 defined by the user interface 40 and distally beyond the vents 80 and HME element 70. The delivery tube 98 may extend through or around the HME element 70. The HME element 70 is shown positioned within the HME cavity 75 that is formed adjacent to but distal from the vents 80. As illustrated in FIGS. 5A and 11, the user interface 40 includes a mask 60 that is cantilevered from a base 48 which engages the user's nares with minimal contact against a user's face. The base is secured to the head of a user with support bands 44.

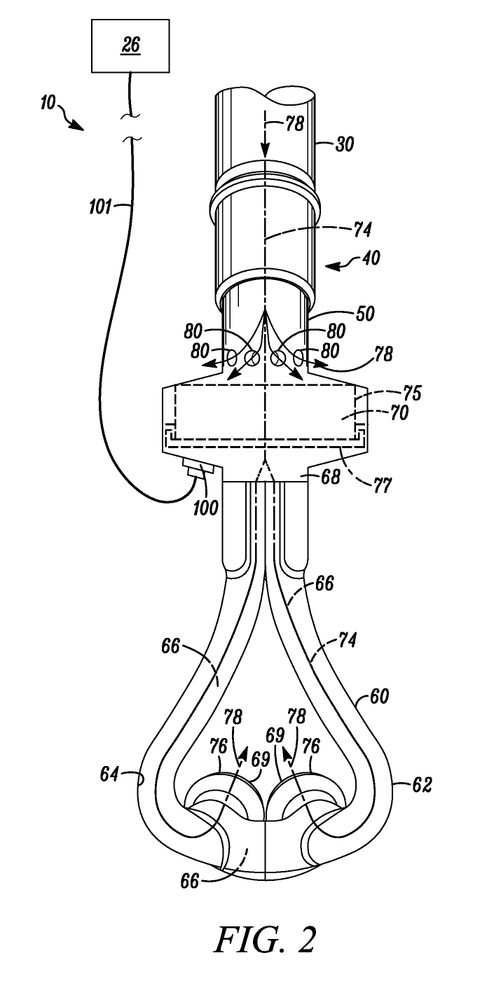

FIG. 2 illustrates another exemplary embodiment of a portion of a user interface 40 of the positive airway pressure therapy apparatus 10. The mask 60 is configured to engage the nares of a user. The air passage 74 extends distally from a delivery tube 30 past vents 80 into an HME cavity 75. The HME cavity 75 is shown with an enlarged diameter relative to the diameter of the adjacent air passage 74. An element lock 77 is provided to secure the HME element 70 in the desired location in the HME cavity 75. A sensor 100 is positioned in communication with a portion of the air passage 74 distal to the HME element 70. The sensor 100 is particularly illustrated as positioned in communication with a distal aspect of the HME cavity 75. The sensor 100 is illustrated in wired communication with the control unit 26. However, the communication of signals between the sensor 100 and control unit 26 could also be in wireless communication through radio frequency, infrared, or other methods as will be recognized by those skilled in the art upon review of the present disclosure. The control unit 26 is shown remotely positioned from the sensor 100. Pressurized air passes along the air passage 74 defined by a distal portion of the user interface 40, through the mask inlet 68, into the mask chamber 66, and exits through mask outlets 69 surrounded by seals 76 into the user's nares. The arrows generally indicative of the flow of pressurized air from the flow generator 20 through the air passage 74, HME element 70 and out the mask outlets 69 representative of an inhalation. Vents 80 are located in the air passage 74 proximal to the HME element 70 for the purging of exhaled CO₂ from the delivery tube 30, The vents 80, in this embodiment, are configured as a series of holes about the circumference of the interface conduit 50 of the user interface 40.

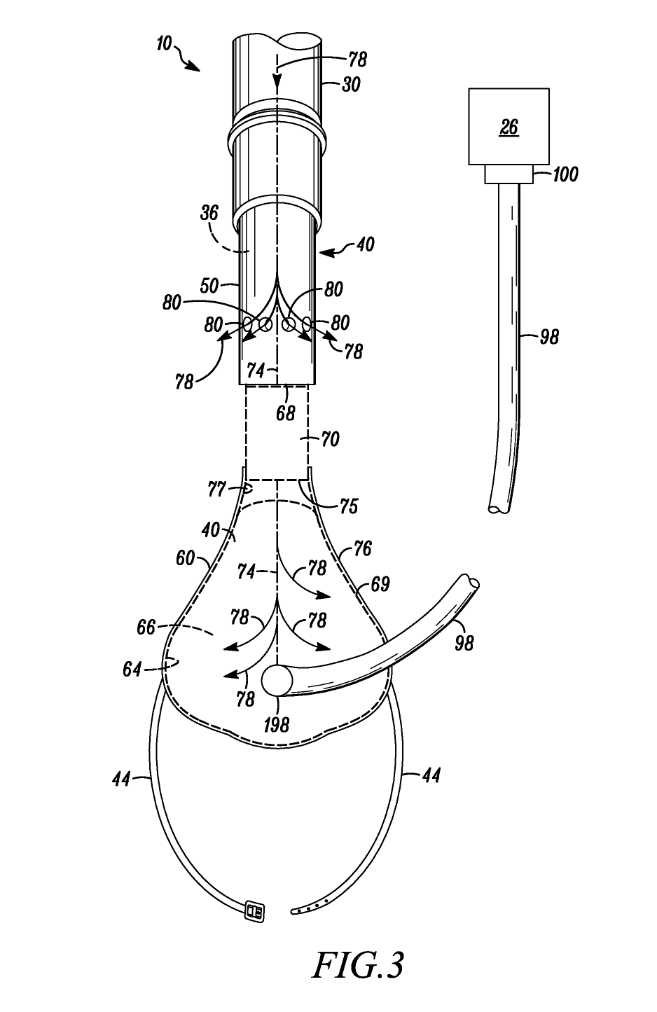

FIG. 3 illustrates another exemplary embodiment of a portion of a user interface 40 of the positive airway pressure therapy apparatus 10. In this embodiment, the sensor tube 98 is positioned externally on the user interface 40. The distal end of the sensor tube 98 is secured in fluid communication with the mask cavity 66 of the mask 60 by a connector 198. The connector 198 may rotate to allow the sensor tube 98 to swivel in order to avoid kinks and twists in the air tube 98. The mask 60, as illustrated, is configured to be secured about the user's nose and mouth so that the user may breathe pressurized air either through the nose or through the mouth. The mask 60 includes the seal 76 around the periphery of the mask 60 to contact and sealingly engage the user's face around both the nose and mouth. Support bands 44 are attached to the mask 60 in this embodiment to secure the mask 60 to the user's face. In this embodiment, the air passage 74 again extends distally from a delivery tube 30 past vents 80 into an HME cavity 75. The HME cavity 75 is shown with a diameter substantially the same as the diameter of the adjacent air passage 74. An element lock 77 in the form of a detent within the air passage 74 is provided to secure the HME element 70 in the desired location in the HME cavity 75. A sensor 100 is positioned remotely from the mask 60. The sensor 100 is in fluid communication with a portion of the mask cavity 66 through the distal end of sensor tube 98. The distal end of the sensor tube 98 communicates with the mask cavity 66. The proximal end of sensor tube 98 communicates with the sensor 100. The sensor 100 is illustrated as integrated into the control unit 26 for exemplary purposes. The control unit 26 and sensor 100 are shown remotely positioned from the user interface 40. Pressurized air passes along the air passage 74 defined by a distal portion of the user interface 40, through the mask inlet 68, into the mask chamber 66, and exits through mask outlet 69 peripherally defined by seal 76 into the user's nose and/or mouth. The arrows generally indicative of the flow of pressurized air from the flow generator 20 through the air passage 74, HME element 70 and out the mask outlets 69 representative of an inhalation. The distal end of the sensor tube 98 permits the monitoring of the pressure in the portion of the air passage 74 distal to the HME element 70 and, particularly as illustrated, in the mask chamber 66. Vents 80 are located in the air passage 74 proximal to the HME element 70 for the purging of exhaled CO₂ that may have accumulated in delivery tube 30. The vents 80, in this embodiment, are configured as a series of holes about the circumference of the interface conduit 50 of the user interface 40.

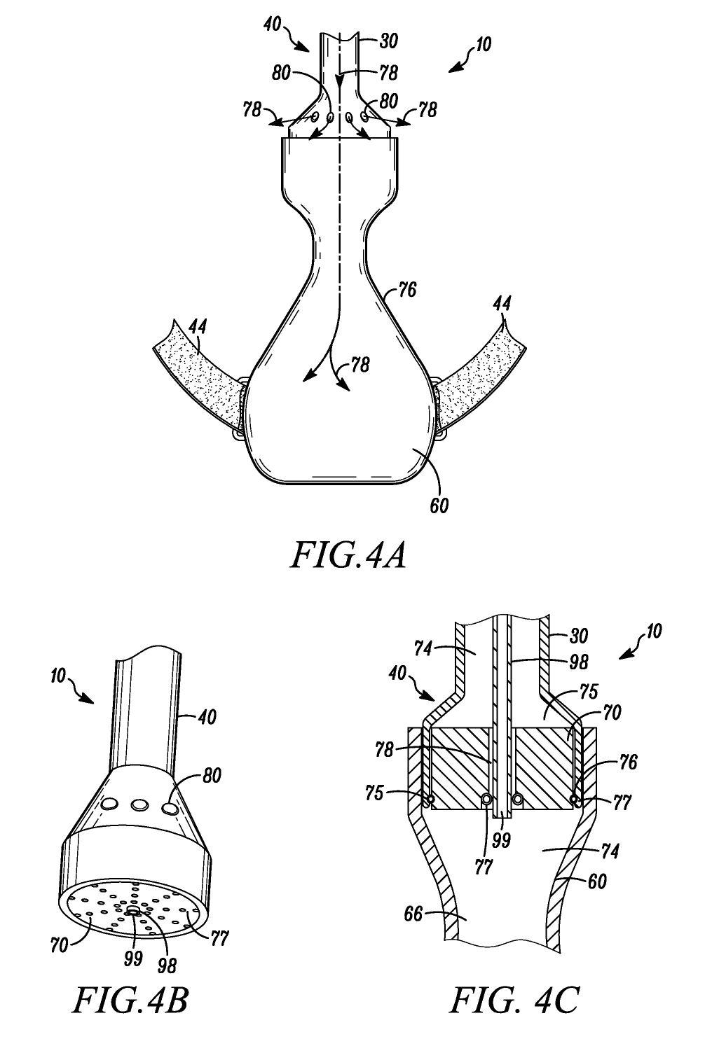

FIGS. 4A to 4C illustrate other exemplary embodiments of a portion of a user interface 40 of the positive airway pressure therapy apparatus 10. In the illustrated embodiments, the sensor tube 98 is positioned internally along at least a portion of the air passage 74. A distal portion of the sensor tube 98 extends through the HME element 70. The distal end of the sensor tube 98 is secured in fluid communication with a portion of the air passage 74 that is distal to HME element 70. Particularly, the sensor tube 98 extends through an HME passage 78. An exterior surface of the sensor tube 98 may sealingly engage the HME passage 78 to prevent air leaks around the HME element. The mask 60, as illustrated, is configured to be secured about the user's nose so that the user may breathe pressurized air through the nose. The mask 60 includes the seal 76, shown in phantom, around the periphery of the mask 60 to contact and sealingly engage the user's face around the nose. Support bands 44 are shown attached to the mask 60 in this embodiment to secure the mask 60 to the user's face. In this embodiment, the air passage 74 again extends distally from a delivery tube 30 out vents 80 to ambient and past vents 80 into an HME cavity 75. The HME cavity 75 is again shown with an enlarged diameter relative the diameter of the adjacent air passage 74. An element lock 77 is illustrated in both FIGS. 4B and 4C in alternative embodiments. In FIG. 4B, the element lock 77 is illustrated as a perforated plate engaged with an inner surface of the HME cavity 75 for exemplary purposes. The HME element 70 is retained in the HME cavity 75 proximal to the perforated plate. In FIG. 4C, the element lock 77 is in the form of an O-ring which is compressionally engaged between an inner wall of the HME cavity 75 and a peripheral surface of the HME element 70 to secure the HME element 70 in the desired location in the HME cavity 75. A second O-ring is illustrated in FIG. 4C as compressionally engaged between the outer surface of air tube 98 and an inner surface of HME element 70. The illustrated O-rings may also function as seals 76 to prevent air leaks around the HME element. A sensor 100, not shown, is positioned remotely from the mask 60. The sensor 100 is in fluid communication with a portion of the air passage 74 distal to the HME element 70 through the distal end of sensor tube 98. The distal end of the sensor tube 98 communicates with the portion of the air passage 74 distal to the HME element 70. The proximal end of sensor tube 98 communicates with the sensor 100. The sensor 100 may be positioned on the flow generator housing 22 or otherwise and is configured to communicate data to the control unit 26, also not shown. Pressurized air passes along the air passage 74 defined by a distal portion of the user interface 40, through the mask inlet 68, into the mask chamber 66, and exits through mask outlet 69 peripherally defined by seal 76 into the user's nose. The arrows 78 generally indicative of the flow of pressurized air from the flow generator 20 through the air passage 74, HME element 70 and out the mask outlets 69 representative of an inhalation. The distal end of the sensor tube 98 permits the monitoring of the pressure in the portion of the air passage 74 distal to the HME element 70. Vents 80 are located in the air passage 74 proximal to the HME element 70 for purging of any exhaled CO₂ that may have accumulated in delivery tube 30. The vents 80, in this embodiment, are configured as a series of holes about the circumference of the interface conduit 50 of the user interface 40.

FIGS. 5A and 5B illustrate another exemplary embodiment of a portion of a user interface 40 of the positive airway pressure therapy apparatus 10. The mask 60 is configured to engage the nares of a user. The air passage 74 extends distally from a delivery tube 30 out vents 80 to ambient and past vents 80 and over an HME element 70. The HME element 70 is secured to an inner mask surface 64 of mask 60. In this embodiment, instead of air passing through the HME element 70, it passes over the surface of the HME element 70. Pressurized air passes along the air passage 74 defined by a distal portion of the user interface 40 and over the HME element 70 until it exits through mask outlets 69 surrounded by seals 76 into the user's nares. Vents 80 are located in the air passage 74 proximal to the HME element 70 for purging of exhaled CO₂ that may have accumulated in delivery tube 30. The vents 80, in this embodiment, are configured as a series of holes about the circumference of the interface conduit 50 of the user interface 40. The HME element 70 may be an insert taking the shape of the outside walls defining the air passage 74 within the interface conduit 50 and/or mask 60. The HME element 70 could be positioned in these portions of the air passage 74 and could be replaced as needed. In another configuration, an HME element 70 lining a portion of the air passage 74 within the interface conduit 50 and/or mask 60 may be configured from a material permitting it to be dried and/or disinfected by placing in the microwave oven or boiled in water for a period of time, by soaking it in disinfectant solution or by other means as will be recognized by those skilled in the art upon review of the present disclosure.

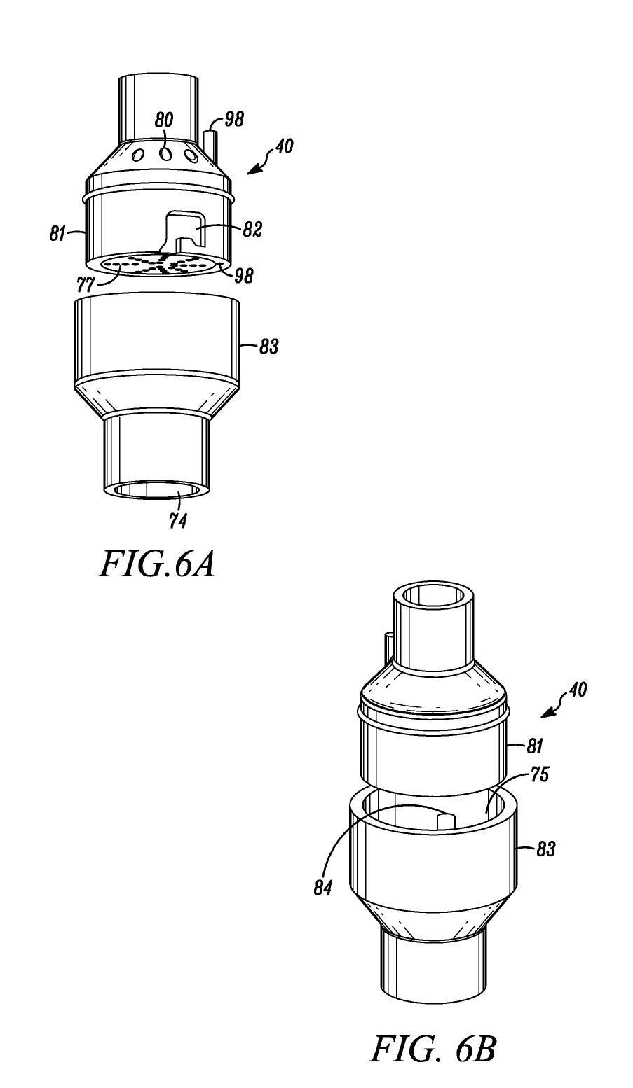

FIGS. 6A and 6B illustrate perspective views of a portion of the user interface 40 defining the HME cavity 77 within the air passage 74 divided into an interlocking upper section 81 and lower section 83. The upper section 81 configured to be removably interlocked with the lower section 83 to permit the replacement of the HME element 70. The upper section 81 defines a locking groove 82 and the lower section 83 defines a locking detent 84 that cooperate to releasably engage the upper section 81 and the lower section 83. FIG. 6A illustrates an elevated perspective view of a portion of the user interface 40 defining the HME cavity 75 within the air passage 74. FIG. 6B illustrates an downward perspective view of a portion of the user interface 40 defining the HME cavity 75 within the air passage 74. The upper section 81 and the lower section 83 are illustrated in a disengaged position for exemplary purposes in both FIGS. 6A and 6B.

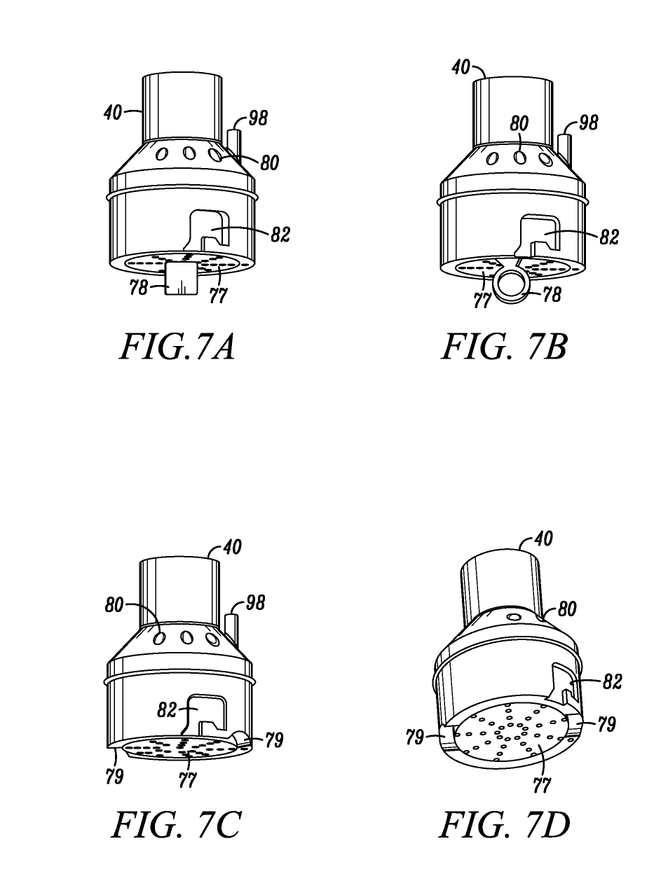

FIG. 7A to 7D illustrates an elevated perspective views of exemplary embodiments of an HME element 70 secured within an HME cavity 75. FIGS. 7A and 7B illustrate an exemplary HME grip 78 to facilitate the removal of the HME element and/or element lock 77. FIGS. 7B and 7D illustrate an exemplary embodiment of a removal notches 79 to facilitate the removal of the HME element and/or element lock 77.

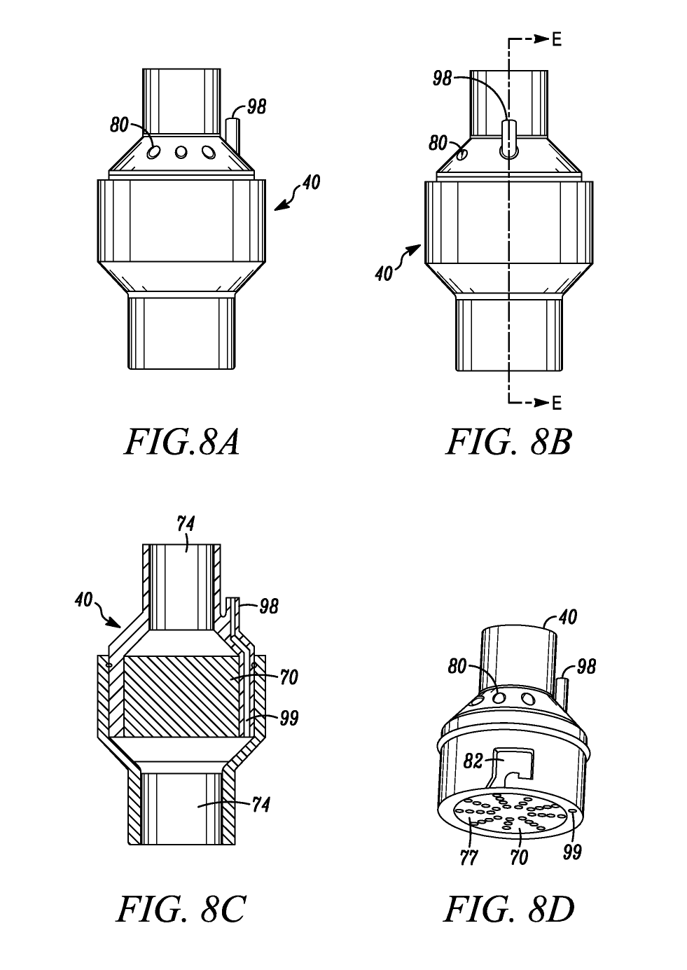

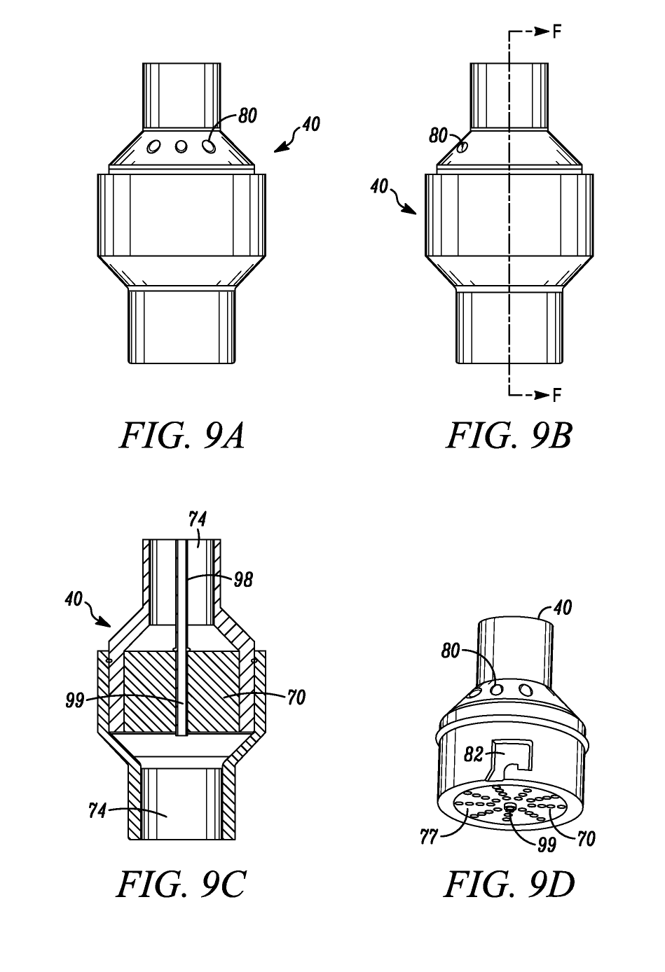

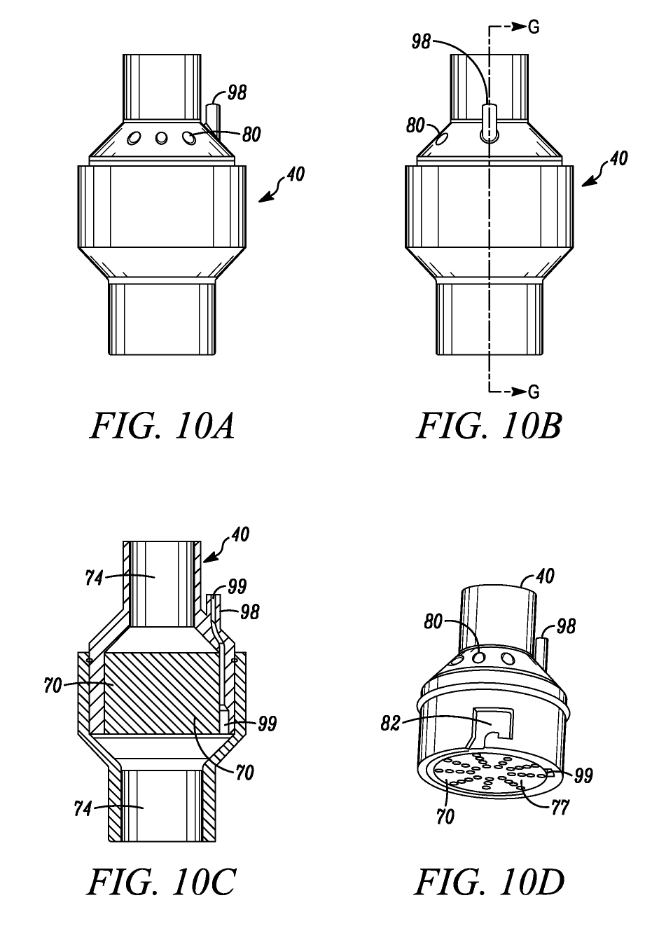

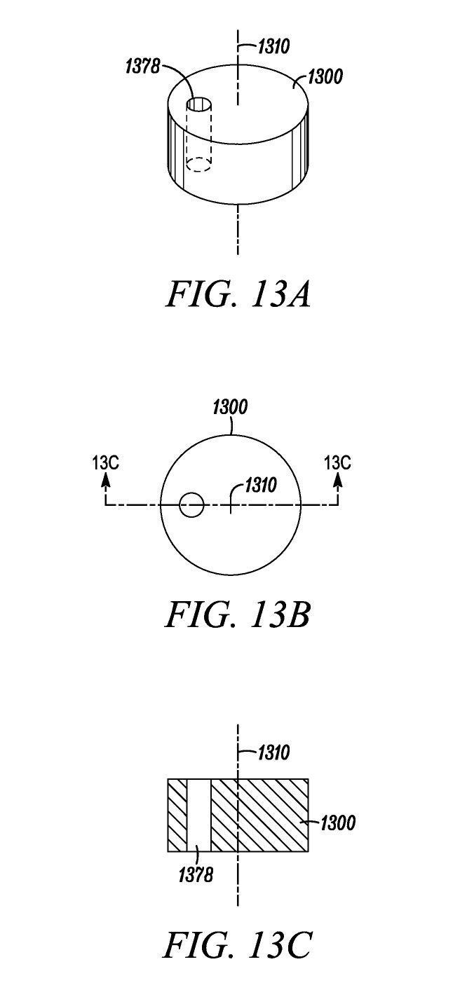

FIGS. 8A to 10D illustrate various views of various exemplary configurations for sensor tube 98 and sensor passage 99 in and around HME element 70. FIG. 13A illustrates an elevated perspective view and FIG. 13B illustrates a top view of an exemplary embodiment of an HME element in accordance with aspects of the present invention. FIG. 13C illustrates cutaway side view along line 13C-13C of FIG. 13B of the HME element in accordance with aspects of the present invention. Now referring to FIGS. 13A-13C, the HME shown will be further detailed. The HME 1300 is configured to be received in a cylindrical opening or HME cavity associated with the positive airway pressure therapy apparatus 10 (shown in FIG. 1). The diameter of the HME 1300 allows the HME 1300 to be in substantially sealing engagement with respect to the HME cavity. The HME element 1300 also has an HME passage 1378 therein for accommodating a sensor, such as pressure sensor tube 98 (shown in FIG. 4C). The diameter of the HME passage 1378 accommodates the sensor tube 98. More particularly, the sensor tube 98 extends through an HME passage 1378, and the exterior surface of the sensor tube 98 sealingly engages the HME passage 1378 to prevent air leaks around the HME element 1300. The HME element 1300 can be formed in many ways. In one example embodiment, the HME element 1300 is formed by attaching an HME material to a core portion and wrapping the material around the core. In another example embodiment, such as the embodiment shown in FIGS. 13A-13C, the HME material is wrapped on itself at the start and then continued. The HME element 1300 that results is cylindrically shaped. In some embodiments, the HME element 1300 may have to be shaped. Once the HME element has the desired shape, the HME passage 1378 is formed by removing HME material from the HME element 1300. The HME passage 1378 can be formed in any desired location, provided there is sufficient HME material to hold the shape of the HME element 1300 and provide a sealing engagement to the sensor tube 98 (shown in FIG. 4C). As shown in FIGS. 13A-13C, the HME passage 1378 is formed at a position parallel to a central axis 1310 of the cylinder and offset from the central axis 1310. It is contemplated that the HME passage 1378 could also be formed non parallel with respect to the central axis 1310 or could be formed so that the axis of the HME passage 1378 is substantially coaxial with respect to the axis 1310. The HME element 1300 can be provided with an element lock (such as element 77 shown in FIG. 4C) or can be shaped to accommodate an element lock positioned within the HME cavity. In another embodiment, the HME may be molded from a foam material. The HME can be made from soft or hard foam materials.

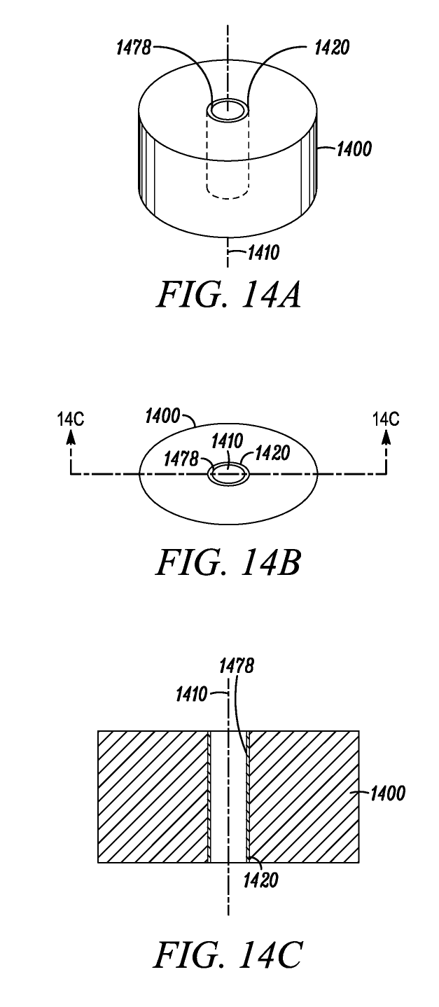

FIG. 14A illustrates an elevated perspective view and FIG. 14B illustrates a top view of an exemplary embodiment of an HME element in accordance with aspects of the present invention. FIG. 14C illustrates cutaway side view along line 14C-14C of FIG. 14B of the HME element in accordance with aspects of the present invention. Now referring to FIGS. 14A-14C, the HME shown will be further detailed. The HME 1400 is configured to be received in a oval opening or HME cavity associated with the positive airway pressure therapy apparatus 10 (shown in FIG. 1). The HME 1400 is shaped to be in substantially sealing engagement with respect to the HME cavity. The HME element 1400 also has an HME passage 1478 therein for accommodating a sensor, such as pressure sensor tube 98 (shown in FIG. 4C). The diameter of the HME passage 1478 accommodates the sensor tube 98. In some embodiments, the sensor tube 98 may be substantially circular in cross section or substantially oval-shaped in cross section. More particularly, the sensor tube 98 extends through an HME passage 1478, and the exterior surface of the sensor tube 98 sealingly engages the HME passage 1478 to prevent air leaks around the HME element 1400. The HME element 1400 can be formed in many ways. In one example embodiment, the HME element 1400 is formed by attaching an HME material to a core portion 1420 and wrapping the material around the core 1420. The core 1420 stays within the HME, as shown in FIGS. 14A-14C. In another embodiment, the core 1420 could be removed. In other words, the core acts as a starting mandrel and is withdrawn. The HME element 1400 that results is oval-shaped or elliptical in cross section. In some embodiments, the HME element 1400 may have to be shaped to achieve the desired cross sectional shape. When the HME passage 1478 is formed by wrapping material around a core or mandrel, the location of the HME opening is generally going to be in a more central location. As shown in FIGS. 14A-14C, the HME passage 1478 is formed at a position parallel to a central axis 1410 of the elliptical cross section (at the intersection of the short axis and the long axis of the ellipse). It is contemplated that the HME passage 1478 could also be formed non parallel with respect to the central axis 1410 or could be formed so that the axis of the HME passage 1478 if the HME 1400 is formed by wrapping the HME material on itself at the start, similar to the HME element 1300 discussed above. The HME element 1400 can be provided with an element lock (such as element 77 shown in FIG. 4C) or can be shaped to accommodate an element lock positioned within the HME cavity. In another embodiment, the HME may be molded from a foam material. The HME can be made from soft or hard foam materials.

The HME element 1300, 1400 is typically made from one or more of three types of materials. These materials may include 1) a hydrophobic material which typically has high antimicrobial properties but may not be as effective in retaining and transferring humidity, 2) a hygroscopic material which typically has much better humidifying effectiveness but may lack microbial filtration properties, and 3) a combination of a hydrophobic material and a hygroscopic material, which may provide both good humidity retention and transfer as well as antimicrobial properties. The material(s) may be formed into various sheets, fibers, foams or other forms which may be arranged in one or more layers or other configurations. The material may be one or more of a cellulose based material, a polysulphone, an electrostatically charged polypropylene, polycarbonate or other materials as will be recognized by those skilled in the art upon review of the present disclosure. In certain aspects, the HME element 1300, 1400 may include an electrostatic filter to assist in the trapping of bacteria and/or viruses. The material may be configured as a set of random or organized intermeshed fibers, a foam or a sheet or in some aspects an array of small parallel tubes. The material can be configured to function as a filter or may include additional filtering components that may be configured to remove particulates including various virus and bacteria.

In certain aspects, the HME element 1300, 1400 may be configured from paper or foam impregnated with a moisture-absorbing hygroscopic salt such as AlCl₃, CaCl₂, MgCl₂, or LiCl which enhances their ability to retain moisture. This configuration of HME element may be referred to as a hygroscopic condenser humidifier. The material may also be treated with various hydrophilic materials such as polyacrylic acid, polyvinyl pyrrolidone, polyvinyl alcohol, or other hydrophilic polymers, glycol or glycerine to enhance its efficiency.

The material is typically incorporated into the HME element 1300, 1400 in a manner which may maximize surface area and reduce resistance to airflow. When formed as a sheet, the material which forms the HME element 70 is typically folded into pleats or rolled to maximize surface area and reduce resistance to the passage of air. However, HME elements 1300 may take a wide variety of forms and be formed from a wide variety of materials and combinations of materials.

In certain aspects, the HME element 1300, 1400 can be formed from one or more layers of aluminum configured for a maximum surface area that comes into contact with the airflow created by respiration in a way that also minimizes resistance to the airflow. On exhalation, moisture condenses on the cooler aluminum sheets while warming the aluminum. On inhalation, the warm, moist aluminum sheets transfer heat and moisture to the incoming air.

In other aspects, the HME element 1300, 1400 can be moisture absorbing heat-insulating disk-like sheets that are alternately interspersed with permeable heat-conductive disk-like sheets. The heat-insulating sheets may be made of gauze or non-woven fabric. The heat-conductive sheets may be made of a mesh of metal such as aluminum. The heat conductive sheets may be spirally wound using aluminum mesh with gauze to allow for the free flow of air across the aluminum winding.

In still other aspects, the HME element 1300, 1400 may be formed from a folded sheet material and strip material where the sheet material is folded with parallel sharp folds with each new fold slightly staggered. The sheet material may be configured to be permeable to air but impermeable to bacteria. Such sheet materials may be either hydrophobic or hydrophilic and may include a matrix of hydrophobized compressed glass fibers. The strip material may be further configured as strips of micro-corrugated paper which is permeable to air and other hydrophilic materials which have good heat and moisture exchange capacity. These materials can, for example, be in the form of foamed polyurethane, loosely packed polyethylene, or polyester fibers. Other materials that may be used can include cardboard or paper, glass fibers, or cellulose within a waved or plane surface. It should be noted, that these and other configurations of the material of HME element 1300, 1400 into any desired shape for fitting within a corresponding HME cavity are contemplated.

The embodiment illustrated in FIG. 11 includes a flow generator 20 that is attached to the user interface 40 generally about the mount 48. A plurality of support bands 44 are provided to secure the user interface 40 including the flow generator 20 about the user's head. Air passage 74 extending from the flow generator housing 22 to the mask 60 is defined by the interface conduit 50 and is maintained in a generally fixed orientation with respect to the user's head. The interface conduit 50 is shown as extending from the flow generator housing 22 and bending to pass over the user's face without touching the user's face and is generally in a fixed orientation with respect to the user's head including the face. The distal end of the interface conduit 50 is secured to the mask 60. Vents 80 are included along the air passage 74 in the interface conduit 50 proximal to the mask 60. The mask 60 is sealed about the user's nares to deliver pressurized air for breathing by the user. An HME element 70 is provide on an inner mask surface 64 of the mask 60. An inner HME surface 72 of the HME element 70 defines at least a portion of the mask chamber 66. The HME element 70 is configured to collect moisture and heat from exhaled air as it passes over the inner HME surface 72 and to at least a portion of the collected moisture and heat into the pressurized air from the flow generator 20 during inhalation. In another embodiment (not shown), a combination of an in-line HME element 70 as shown in FIGS. 2-4 with an HME element 70 lining at least a portion of the interface and interface conduit may be employed.

FIG. 12 illustrates an exemplary block diagram for the control and regulation of a positive airway pressure therapy apparatus 10 in accordance with the present inventions. As illustrated, the flow generator 20, the control interface 28, and the sensor 100 are in communication with the control unit 26 to permit a signal to be communicated to the control unit 26. The control unit 26 is in communication with each of the flow generator 20 and the control interface 28 to permit a signal to be communicated to the flow generator 20 or the control interface 28. In an exemplary state of operation, a therapy may be initiated by a user through an input to the control interface 28. The control interface 28 may communicate a signal to the control unit to initiate a therapy for the desired therapy. The control unit 26 provides a control signal to the flow generator 20 to deliver the desired physical parameters of the positive airway pressure therapy to the user. The control unit 26 receives a signal indicative of one or more of pressure and airflow at a location in the air passage 74 (not shown) distal to an HME element 70 (also not shown) from the sensor 100. The control unit 26 interprets the signal and compares the signal with a desired value. If outside the desired value, the control unit 26 communicates a control signal to the flow generator 20 to adjust the therapy to the desired physical parameters.

The foregoing discussion discloses and describes merely exemplary embodiments of the present inventions. Upon review of the specification, one skilled in the art will readily recognize from such discussion, and from the accompanying figures and claims, that various changes, modifications and variations can be made therein without departing from the spirit and scope of the invention as defined in the following claims.

1. A positive airway pressure therapy apparatus, comprising:
a flow generator, the flow generator configured to provide pressurized air to an air passage, the flow generator configured for administration of a positive pressure airway therapy to a user at a pressure of at least between 4 and 20 centimeters of water;
a user interface, the user interface including at least one vent, an HME element, a mask and support bands, the user interface defining at least a portion of the air passage, the at least one vent in communication with the air passage to communicate air between at least the air passage and ambient atmosphere, the HME element secured within the air passage at a location distal to the at least one vent, the mask defining a mask outlet configured to communicate the pressurized air to an airway of the user, the outlet positioned distally along the air passage from the HME element; and
an HME grip associated with the HME element, the HME grip operable to facilitate removal of the HME element from the air passage.

2.-16. (canceled)

17. A positive airway pressure therapy apparatus, comprising:
a flow generator configured to provide pressurized air, via an air passage, to an airway of a user during delivery of a positive airway pressure therapy; and
a user interface defining at least a portion of the air passage, the user interface comprising:
a mask defining a mask outlet in communication with the air passage, the mask outlet configured to communicate the pressurized air to the airway of the user;
at least one vent in communication with the air passage and configured to communicate air between the air passage and ambient atmosphere;
a heat and moisture exchanger (HME) element secured within an HME cavity formed within the air passage at a location between the at least one vent and the mask outlet; and
an HME element lock configured to secure the HME element relative to the HME cavity.

18. The apparatus of claim 17, further comprising an HME grip to facilitate removal of the HME element from the HME cavity.

19. The apparatus of claim 17, wherein the HME element lock comprises a perforated plate engagable with a surface of the HME cavity.

20. The apparatus of claim 17, wherein the HME element lock comprises one or more O-rings compressionally engaged between the HME cavity and the HME element.

21. The apparatus of claim 17, further comprising one or more support bands configured to secure both the user interface and the flow generator about a head of the user.

22. The apparatus of claim 17, wherein the HME cavity defines an elliptical cross section.

23. The apparatus of claim 17, wherein the HME element comprises a material selected from the group consisting of hydrophobic materials, hygroscopic materials, and a combination of hydrophobic materials and hydroscopic materials.

24. The apparatus of claim 17, wherein the HME element comprises an antimicrobial or electrostatic material.

25. The apparatus of claim 17, wherein the HME element defines an opening configured to permit a sensor tube to pass through the HME element.

26. The apparatus of claim 17, wherein the air passage is defined at least in part by a delivery tube comprising a proximal end attached to an outlet of the flow generator, and a distal end attached to an inlet of the mask.

27. A positive airway pressure therapy apparatus, comprising:
a flow generator configured to provide pressurized air, via an air passage, to an airway of a user during delivery of a positive airway pressure therapy; and
a user interface defining at least a portion of the air passage, the user interface comprising:
a mask defining a mask outlet in communication with the air passage, the mask outlet configured to communicate the pressurized air to the airway of the user;
a vent in communication with the air passage to communicate air between the air passage and ambient atmosphere;
a heat and moisture exchanger (HME) element secured within an HME cavity formed within the air passage at a location between the vent and the mask outlet; and
an HME grip associated with the HME element, the HME grip operable to facilitate removal of the HME element from the HME cavity.

28. The apparatus of claim 27, wherein the air passage is defined at least in part by a delivery tube comprising a proximal end, which is attached to the flow generator, and a distal end, which is attached to the mask.

29. The apparatus of claim 28, wherein the HME cavity is formed at least in part by the delivery tube.

30. The apparatus of claim 27, wherein the HME cavity is located adjacent the vent.

31. The apparatus of claim 27, wherein the HME cavity has a diameter larger than a diameter of an adjacent portion of the air passage.

32. A user interface for use with a positive airway pressure therapy apparatus, the user interface comprising:
a mask defining: a mask inlet for receiving pressurized air from a flow generator; and a mask outlet for delivering the pressurized air to an airway of a user, wherein an air passage is formed and extends from the flow generator to the mask outlet;
at least one vent in communication with the air passage to communicate air between the air passage and ambient atmosphere;
a heat and moisture exchange (HME) element secured within an HME cavity, the HME cavity formed within the air passage between the at least one vent and the mask outlet; and
an HME grip associated with the HME element, the HME grip operable to facilitate removal of the HME element from the HME cavity.

33. The user interface of claim 32, further comprising one or more support bands configured to secure the mask to the user.

34. The user interface of claim 32, further comprising a delivery tube having a distal end coupled to the mask, the delivery tube forming at least a part of the air passage, wherein the at least one vent is located near the distal end of the delivery tube.

35. The user interface of claim 32, further comprising an HME element lock configured to secure the HME element within the HME cavity.

36. The user interface of claim 32, wherein the HME cavity has a cross-sectional dimension larger than a cross-sectional dimension of an adjacent portion of the air passage.
